(19) United States
(12) Patent Application Publication
(10) Pub. No.: US 2024/0041766 A1
(43) Pub. Date: Feb. 8, 2024

(54) TOPICAL PHARMACEUTICAL COMPOSITION COMPRISING AMITRIPTYLINE AND AN ALKALINE AQUEOUS PHASE

(71) Applicant: AlgoTherapeutix, Suresnes (FR)

(72) Inventors: Frédéric Lallemand, Rambouillet (FR); Philippe PICAUT, Verrieres Le Buisson (FR)

(73) Assignee: AlgoTherapeutix, Suresnes (FR)

(21) Appl. No.: 18/550,908

(22) PCT Filed: Mar. 15, 2022
(86) PCT No.: PCT/FR2022/050461

§ 371 Date: Sep. 15, 2023

(30) Foreign Application Priority Data
Mar. 19, 2021 (FR) FR2102760

(51) Int. Cl. A61K 9/107; A61K 9/00; A61K 47/38; A61K 31/135

(52) U.S. Cl. CPC A61K 9/107; A61K 9/0014; A61K 47/38; A61K 31/135

(57) ABSTRACT

The present invention relates to a topical pharmaceutical composition in the form of an oil-in-water emulsion comprising an oily phase based on amitriptyline in its basic form and an alkaline aqueous phase.

The invention also relates to a method for treating neuropathic pain or erythromelalgia comprising the topical application to the patient of a pharmaceutical composition according to the invention.

The present invention relates to a topical pharmaceutical composition in the form of an oil-in-water emulsion comprising an oily phase based on amitriptyline in its basic form and an alkaline aqueous phase.

The invention also relates to a method for treating neuropathic pain or erythromelalgia comprising the topical application to the patient of a pharmaceutical composition according to the invention.

Peripheral neuropathic pain is caused by damage to nerve structures such as peripheral nerve endings or nociceptors which become extremely sensitive to stimulation and which could generate impulses in the absence of stimulation.

This damage could be caused for many reasons like trauma, diseases such as diabetes, shingles and advanced cancers, chemotherapeutic treatments or a chemical burn. A lesion of the peripheral nerve could lead to pathological conditions characterised by the presence of superficial (sensation of burning or painful cold) or deep (sensation of compression or vice) continuous spontaneous pain, paroxysmal pain (electrical shocks, stab) with, on clinical examination, hypoesthesia or, on the contrary, hyperalgesia (increased response to noxious stimuli), allodynia (pain induced by a non-painful stimulus) or hyperpathia (persistent pain during ordinarily repeated non-nociceptive stimulations). Neuropathies could also be associated with sensory signs such as paraesthesia, numbness, and pruritus.

Chemo-induced neuropathies are particularly frequent, disabling and difficult to treat. They are dose-dependent. Peripheral nerve damages represent most of neurological damages related to chemotherapy toxicity. They are the consequence of a direct toxic damage to the axon or a demyelination and represent the most frequent limiting factor after haematological toxicity.

The application WO 2018/197307 previously filed by Algotherapeutix, as well as the article “Rossignol, J. et al. High concentration of topical amitriptyline for treating chemotherapy-induced neuropathies. Support Care Cancer 27, 3053-3059 (2019)” describe the topical use in the treatment of peripheral neuropathic pain of pharmaceutical compositions in the form of a cream with a high amitriptyline concentration and a slightly acidic pH.

The application FR 2 003 425 (registration number) previously filed by Algotherapeutix, describes the topical use in the treatment of peripheral neuropathic pain of a pharmaceutical composition in the form of a gel with a high amitriptyline concentration and an acidic pH.

There is a real need to develop and provide new compositions based on amitriptyline, which have a good effectiveness, and possibly an improved effectiveness, in cutaneous application in the treatment of pain, in particular in the treatment of peripheral neuropathic pain and in particular chemotherapy-induced neuropathic pain.

Furthermore, amitriptyline, like all tricyclic antidepressants, could have many undesirable effects (arterial hypotension, sedation, QT prolongation), in particular when administered orally.

Thus, it is also interesting to formulate new compositions with a low amitriptyline concentration, in order to reduce the risk of undesirable effects, while maintaining a good effectiveness, and possibly improving the effectiveness, in cutaneous application in the treatment of pain.

It has been surprisingly discovered that a pharmaceutical composition for topical application in the form of an oil-in-water emulsion comprising an oily phase containing 1% to 30% by weight of amitriptyline in its basic form with respect to the total weight of the composition, and an aqueous phase with a pH higher than or equal to 7, allowed effectively treating pain, in particular chemotherapy-induced peripheral neuropathic pain (or CIPN, standing for “chemotherapy-induced peripheral neuropathy”), post-herpetic neuropathic pain (or PHN, standing for “post herpetic neuralgia”) or diabetic neuropathic pain (or DPN, standing for “diabetic peripheral neuropathy”).

In particular, this discovery is even more surprising since, contrary to what was expected, increasing the pH of a composition based on amitriptyline at an alkaline pH did not have the effect of precipitating the amitriptyline. The composition is then in the form of an emulsion comprising droplets of amitriptyline in its basic form dispersed in an alkaline aqueous phase.

Thus, an object of the invention is a pharmaceutical composition in the form of an oil-in-water emulsion for topical application comprising:
(i) an oily phase containing at least amitriptyline in its basic form, in a total content ranging from 1% to 30% by weight with respect to the total weight of the composition; and
(ii) an aqueous phase, having a pH higher than or equal to 7.

It has been noticed that the pharmaceutical composition according to the invention allows facilitating the penetration of amitriptyline through the skin, and thus obtaining a good therapeutic effectiveness, even with a low amitriptyline concentration.

In particular, it has been observed that the composition according to the invention with an aqueous phase having a pH higher than or equal to 7, and more preferably at a pH higher than or equal to 8, still better at a pH between 8.5 and 12, features a better penetration of amitriptyline through the skin than a similar composition with an aqueous phase at acidic pH.

Moreover, the composition according to the invention has a good bioavailability at amitriptyline concentrations preferably between 1% to 10% by weight and more preferably between 1% and 9% by weight, with respect to the total weight of the composition.

Furthermore, the composition according to the invention comprises few excipients, which promotes a good local tolerance of the composition (less risk of allergy, less risk of irritation).

The composition according to the invention also has good usage properties, namely the composition is odourless and pleasant to the touch.

In a particularly surprising manner, it has also been discovered that the pharmaceutical composition according to the invention allowed an effective topical treatment of erythromelalgia.

Erythromelalgia is an uncommon episodic acrosyndrome primarily affecting both lower limbs symmetrically by the presence of erythema, heat and burning pain. Many scientific articles describe this orphan disease, in particular “Leroux MB. Erythromelalgia: a cutaneous manifestation of neuropathy? An Bras Dermatol. 2018; 93(1): 86-94”.

Topical (cutaneous) application of the composition according to the invention results in an effective treatment of erythromelalgia and neuropathic pain, more particularly of peripheral neuropathic pain such as chemotherapy-induced, post-herpetic, and diabetic peripheral neuropathic pain.

It has been noticed that, besides alleviating pain, a composition based on amitriptyline also allows restoring a healthier skin.

In addition, the topical application of the composition according to the invention has few, and even no, side effects. In particular, no cutaneous irritation is observed at the place where the composition is applied.

Another object of the invention is a method for treating neuropathic pain, such as peripheral neuropathic pain, or erythromelalgia comprising the topical application of a pharmaceutical composition according to the invention.

Other objects, features, aspects and advantages of the invention will appear even more clearly upon reading the description and the following example.

In the present description, and unless indicated otherwise:
the expression “at least one” is equivalent to the expression “one or more” and could be substituted therefor;
the expression “comprised between . . . and . . . ” is equivalent to the expression “ranging from . . . to . . . ” and could be substituted therefor, and implies that the bounds are included;
the expression “polyoxyalkylenated” corresponds, in the context of the invention, to a —(O-alkyle)ₙ- unit, where n is an integer varying from 2 to 200, preferably from 2 to 40, more preferably from 2 to 20;
the expression “polyoxyethylenated” corresponds, in the context of the invention, to a —(O—CH₂CH₂)ₙ—, where n is an integer varying from 2 to 200, preferably from 2 to 40, more preferably from 2 to 20.

According to the invention, the composition is in the form of an oil-in-water emulsion. The composition according to the invention is not in the form of a gel.

Preferably, the oil-in-water emulsion according to the invention has an average volume size of the oily droplets ranging from 1 nm to 50 μm.

More preferably, the average volume size of the oily droplets in the composition according to the invention ranges from 10 nm to 20 μm, and even more preferably from 100 nm to 10 μm.

In particular, the average volume size of the oily droplets may be determined using the known dynamic light scattering (DLS) method. As a device that can be used for this determination, mention may be made of a Zeiss brand, Axio model microscope or the Malvem brand, Mastersizer 3000 model or Zetasizer Nano ZS model granulometers, equipped with a standard laser with a 4 mW power and at a 633 nm wavelength. This device is also equipped with a correlator (25 ns to 8,000 s, 4,000 channels maximum).

Amitriptyline

The composition according to the present invention comprises an oily phase based on amitriptyline in its basic form.

According to the invention, the total amitriptyline content ranges from 1% to 30% by weight with respect to the total weight of the composition.

Amitriptyline has the following formula (I):

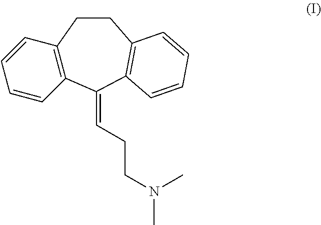 [SMILES: CN(C)CCC=C1c2ccccc2CCc2ccccc21]

Advantageously, the amitriptyline present in the composition according to the invention is in its basic form. In other words, the amitriptyline present in the composition according to the invention is not salified. Still again, the nitrogen atom of amitriptyline present in the composition according to the invention is not protonated.

Preferably, amitriptyline in its basic form constitutes the oily phase of the composition according to the invention.

According to a particular embodiment of the invention, the composition according to the invention may further comprise one or more liquid fatty substance(s), other than amitriptyline.

Preferably, the total content of amitriptyline ranges from 1% to 10% by weight, more preferably from 1% to 9% by weight, even more preferably from 1% to 8% by weight, and still better from 1% to 7% by weight, with respect to the total weight of the composition.

In particular, a better penetration of amitriptyline through the skin has been observed, when the total content of amitriptyline ranges from 1% to 10% by weight, better from 1% to 9% by weight, still better from 1% to 8% by weight, and even better from 1% to 7% by weight, with respect to the total weight of the composition according to the invention.

Water

The composition according to the present invention comprises water.

Preferably, the total water content is greater than or equal to 75% by weight, more preferably comprised between 75% and 95% by weight; even more preferably between 75% and 90% by weight, with respect to the total weight of the composition.

Surfactants

Preferably, the composition according to the present invention further comprises at least one surfactant.

The surfactants that can be used according to the invention may be chosen from anionic surfactants, cationic surfactants, amphoteric or zwitterionic surfactants, non-ionic surfactants, and mixtures thereof.

More preferably, the surfactant(s) that can be used according to the invention are chosen from non-ionic surfactants.

The non-ionic surfactants that can be used according to the invention may be chosen from alkyl polyglucosides (APG), oxyalkylenated glycerol esters, oxyalkylenated fatty acid and sorbitan esters, polyoxyalkylenated fatty acid esters (in particular polyoxyethylenated and/or polyoxypropylenated) optionally in combination with a fatty acid and glycerol ester like the PEG-100 Stearate/Glyceryl Stearate mixture marketed for example by the company ICI under the name Arlacel 165, oxyalkylenated sugar esters, and mixtures thereof.

As alkylpolyglucosides, mention may be made of those containing an alkyl group including from 6 to 30 carbon atoms and preferably from 8 to 16 carbon atoms, and containing a glucoside group preferably comprising 1.2 to 3 glucoside units. For example, the alkylpolyglucosides may be selected from decylglucoside (Alkyl-C₉/C₁₁-polyglucoside (1.4)) like the product marketed under the name Mydol 10° by the company Kao Chemicals or the product marketed under the name Plantacare 2000 UP® by the company Cognis; caprylyl/capryl glucoside like the product marketed under the name Plantacare KE 3711° by the company Cognis; laurylglucoside like the product marketed under the name Plantacare 1200 UP ° by the company Cognis; cocoglucoside like the product marketed under the name Plantacare 818 UP ° by the company Cognis; caprylylglucoside like the product marketed under the name Plantacare 810 UP ° by the company Cognis; and mixtures thereof.

The polyoxyalkylenated glycerol esters are in particular the polyoxyethylenated derivatives of glyceryl and fatty acid esters and of their hydrogenated derivatives. These oxyalkylenated glycerol esters can be chosen, for example, from esters of glyceryl and hydrogenated and oxyethylenated fatty acids, such as PEG-200 hydrogenated glyceryl palmate marketed under the name Rewoderm LI-S 80 by the company Goldschmidt; oxyethylenated glyceryl cocoates such as PEG-7 glyceryl cocoate marketed under the name Tegosoft GC by the company Goldschmidt, and PEG-30 glyceryl cocoate marketed under the name Rewoderm LI-63 by the company Goldschmidt; oxyethylenated glyceryl stearates; and mixtures thereof.

In particular, the oxyalkylenated sugar esters are polyethylene glycol ethers of fatty acid and sugar esters. For example, these oxyalkylenated sugar esters may be chosen from oxyethylenated glucose esters such as PEG-120 methyl glucose dioleate marketed under the name Glucamate DOE 120 by the company Amerchol.

Preferably, the number of moles of alkylene oxide of the non-ionic surfactants that can be used according to the invention varies from 2 to 400; more preferably from 4 to 250.

Preferably, the composition according to the invention comprises at least one non-ionic surfactant; more preferably a non-ionic surfactant, optionally polyoxyalkylenated, chosen from sorbitan esters, glycerol esters, and mixtures thereof; even more preferably from among polyoxyalkylenated glycerol esters; even better from among hydrogenated and polyoxyethylenated glyceryl esters and fatty acids, polyoxyethylenated glyceryl cocoates, polyoxyethylenated glyceryl stearates, and mixtures thereof.

Preferably, when the composition according to the invention comprises at least one surfactant, the total content of surfactant(s) is comprised between 0.1% and 10% by weight, more preferably between 0.5% and 5% by weight, even more preferably between 1% and 4% by weight, with respect to the total weight of the composition.

Preferably, when the composition according to the invention comprises at least one surfactant, the total content of non-ionic surfactant(s) is comprised between and 10% by weight, more preferably comprised between 0.5% and 5% by weight, even more preferably between 1% and 4% by weight, with respect to the total weight of the composition.

Polyols

Preferably, the composition according to the present invention further comprises at least one C₂-C₈ polyol.

In the context of the present invention, by “C₂-C₈ polyol”, it should be understood an organic compound consisting of a C₂-C₈ hydrocarbon chain, optionally interrupted by one or more oxygen atom(s), and bearing at least two free hydroxyl groups (—OH) borne by different carbon atoms, this compound could be cyclic or acyclic, linear or branched, saturated or unsaturated, and in the liquid state at room temperature (25° C.) and at atmospheric pressure (namely 1.013×10⁵ Pa).

Preferably, the C₂-C₈ polyol(s) according to the invention is/are acyclic and non-aromatic.

The C₂-C₈ polyols according to the invention comprise in their structure from 2 to 8 carbon atoms, preferably from 2 to 6 carbon atoms, more preferably from 2 to 5 carbon atoms.

More particularly, the polyol(s) that can be used according to the invention comprise from 2 to 10 hydroxy groups, more preferably from 2 to 5 hydroxy groups, more preferably from 2 to 3 hydroxy groups.

Preferably, said C₂-C₈ polyol(s) which can be used according to the invention are chosen from C₃-C₆ polyols, ethylene glycol, and mixtures thereof.

According to a preferred embodiment of the invention, said C₂-C₈ polyol(s) which can be used according to the invention are chosen from propylene glycol, 1,3-propanediol, 1,3-butylene glycol, pentane-1,2-diol, dipropylene glycol, hexylene glycol, pentylene glycol, glycerol, ethylene glycol, and a mixture of these compounds; more preferably the composition comprises at least propylene glycol.

Preferably, when the composition according to the invention comprises at least one C₂-C₈ polyol, the total content of C₂-C₈ polyol(s) is comprised between 0.1 and 15% by weight, more preferably between 0.5 and 10% by weight, even more preferably between 1 and 6% by weight, and still better between 3 and 6% by weight, with respect to the total weight of the composition.

Preferably, when the composition according to the invention comprises at least one C₂-C₈ polyol, the total propylene glycol content is comprised between 0.1 and 15% by weight, more preferably between 0.5 and 10% by weight, even more preferably between 1 and 6% by weight, and still better between 3 and 6% by weight, with respect to the total weight of the composition.

Thickening Agents

Preferably, the composition according to the invention further comprises at least one thickening agent.

More preferably, the thickening agent(s) is/are thickening polymers.

According to the present invention, by “thickening polymer”, it should be understood polymers that increase, by their presence at a concentration of 0.05% by weight, the viscosity of the cosmetic compositions into which they are introduced by at least 20 cps (20 mPa·s), preferably at least 50 cps (50 mPa·s), at room temperature (25° C.), at atmospheric pressure and at a shear rate of 1 s⁻¹ (the viscosity may be measured using a cone/plane viscometer, a Haake R600 Rheometer or the same).

As an example, as a thickening agent, mention may be made of: crosslinked homopolymers or copolymers of acrylic or methacrylic acid, crosslinked homopolymers of 2-acrylamido-2-methyl-propane sulphonic acid and their crosslinked acrylamide copolymers, homopolymers of ammonium acrylate or copolymers of ammonium acrylate and acrylamide, cellulose polymers, and mixtures thereof.

Among crosslinked homopolymers of acrylic acid, mention may be made of those crosslinked with an alcohol allyl ether of the sugar series, like for example the products sold under the names CARBOPOLS 980, 981, 954, 2984 and 5984 by the company NOVEON or the products sold under the names SYNTHALEN M and SYNTHALEN K by the company 3 VSA. These polymers have the INCI name Carbomer. The thickening polymers may also be crosslinked copolymers of (meth)acrylic acid such as the polymer sold under the name AQUA SF1 by the company NOVEON.

Even more preferably, the composition according to the invention further comprises at least one cellulose polymer.

According to the invention, by “cellulosic” polymer, it should be understood any polysaccharide compound, substituted or not, having in its structure chains of glucose residues united by (3-1,4 bonds; besides unsubstituted celluloses, the cellulose derivatives may be anionic, cationic, amphoteric or non-ionic.

Thus, the cellulose polymers that can be used according to the invention may be chosen from unsubstituted celluloses, including in a microcrystalline form, and substituted celluloses.

More preferably, the cellulosic polymers that can be used according to the invention do not include a C₁₀-C₃₀ fatty lateral chain in their structure.

Preferably, the cellulosic polymer(s) that can be used according to the invention have an average molecular weight comprised between 5,000 and 1,500,000, more preferably between 50,000 and 800,000, even more preferably between 400,000 and 800,000.

Among the cellulose polymers according to the invention, it is possible to distinguish cellulose ethers, cellulose esters and cellulose ether esters.

Among cellulose esters, mention may be made of inorganic cellulose esters (cellulose nitrates, sulphates or phosphates . . . ), organic cellulose esters (cellulose monoacetates, triacetates, amidopropionates, acetatebutyrates, acetatepropionates or acetatetrimellitates . . . ) and mixed organic/inorganic cellulose esters such as cellulose acetatebutyrate sulphates and cellulose acetatepropionate sulphates. Among cellulose ether esters, mention may be made of hydroxypropylmethyl cellulose phthalates and ethyl cellulose sulphates.

Among non-ionic cellulose ethers, mention may be made of (C₁-C₄)alkyl celluloses such as methyl celluloses and ethyl celluloses (for example Ethocel standard 100 Premium from DOW CHEMICAL); (poly)hydroxy(C₁-C₄)alkyl celluloses such as hydroxymethyl celluloses, hydroxyethyl celluloses (for example Natrosol 250 HHR offered by AQUALON) and hydroxypropyl celluloses (for example Klucel EF from AQUALON); mixed (poly)hydroxy(C₁-C₄)alkyl-(C₁-C₄)alkyl celluloses such as hydroxypropyl-methyl celluloses (for example Methocel E4M from DOW CHEMICAL), hydroxyethyl-methyl celluloses, hydroxyethyl-ethyl celluloses (for example Bermocoll E 481 FQ from AKZO NOBEL) and hydroxybutyl-methyl celluloses.

Among anionic cellulose ethers, mention may be made of (poly)carboxy(C₁-C₄)alkyl celluloses and salts thereof. As an example, mention may be made of carboxymethyl celluloses, carboxymethylmethyl celluloses (for example Blanose 7M from the company AQUALON) and carboxymethylhydroxyethyl celluloses and sodium salts thereof.

Among cationic cellulose ethers, mention may be made of cationic cellulose derivatives such as cellulose copolymers or cellulose derivatives grafted with a water-soluble quaternary ammonium monomer, and described in particular in the patent U.S. Pat. No. 4,131,576, such as (poly)hydroxy(C₁-C₄)alkyl celluloses, like hydroxymethyl-, hydroxyethyl- or hydroxypropyl celluloses grafted in particular with a methacryloylethyl-trimethylammonium, methacrylmidopropyl-trimethyl ammonium or dimethyl-diallylammonium salt. More particularly, the products marketed meeting this definition are the products sold under the name “Celquat®L 200” and “Celquat®H 100” by the Company National Starch.

According to a preferred embodiment of the invention, the cellulosic polymer(s) are chosen from cellulosic polymers which do not include a C₁₀-C₃₀ fatty lateral chain in their structure; more preferably from among cellulose ethers; even more preferably from among non-ionic cellulose ethers; still better from among (a) (C₁-C₄)alkyl celluloses such as methyl celluloses and ethyl celluloses, (b) (poly)hydroxy(C₁-C₄)alkyl celluloses such as hydroxymethyl celluloses, hydroxyethyl celluloses and hydroxypropyl celluloses, (c) mixed (poly)hydroxy(C₁-C₄)alkyl-(C₁-C₄)alkyl celluloses such as hydroxypropyl-methyl celluloses, hydroxypropyl-ethyl celluloses, hydroxyethyl-methyl celluloses, hydroxyethyl-ethyl celluloses and hydroxybutyl-methyl celluloses, and (d) mixtures thereof.

More preferentially, the composition according to the invention comprises at least one (poly)hydroxy(C₁-C₄)alkyl cellulose such as hydroxymethyl celluloses, hydroxyethyl celluloses and hydroxypropyl celluloses; still better at least hydroxyethyl cellulose.

Preferably, when the composition according to the invention comprises at least one thickening agent, the total content of thickening agent(s) is comprised between 0.1% and 10% by weight, more preferably between 0.5% and 5% by weight, even more preferably between 1% and 2.5% by weight, with respect to the total weight of the composition.

Preferably, when the composition according to the invention comprises at least one cellulosic polymer, the total content of cellulosic polymer(s) is comprised between 0.1% and 10% by weight, more preferably between 0.5% and 5% by weight, even more preferably between 1% and 2.5% by weight, with respect to the total weight of the composition.

Preferably, when the composition according to the invention comprises at least one cellulosic polymer, the total content of (poly)hydroxy(C₁-C₄)alkyl cellulose(s) is comprised between 0.1% and 10% by weight, more preferably between and 5% by weight, even more preferably between 1% and 2.5% by weight, with respect to the total weight of the composition.

Preferably, when the composition according to the invention comprises at least one cellulosic polymer, the total content of hydroxyethyl cellulose is comprised between 0.1% and 10% by weight, more preferably between 0.5% and 5% by weight, even more preferably between 1% and 2.5% by weight, with respect to the total weight of the composition.

Liquid Fatty Substances

The composition according to the invention may possibly further comprise at least one liquid fatty substance.

In the context of the present invention, by “fatty substance”, it should be understood an organic compound insoluble in water at 30° C. and at atmospheric pressure (760 mm Hg, or 1.013×10⁵ Pa), i.e. with a solubility lower than 5% and preferably lower than 1%, even more preferably lower than 0.1% (gram per mL of water).

In general, liquid fatty substances are soluble in organic solvents under the same temperature and pressure conditions, like for example chloroform, ethanol or benzene.

In the context of the present invention, by “liquid fatty substances”, it should be understood a fatty substance in the liquid state at 25° C. and at atmospheric pressure (760 mm Hg, or 1.013×10⁵ Pa).

Preferably, they have a viscosity lower than or equal to 2 Pa·s, still better lower than or equal to 1 Pa·s and even better lower than or equal to 0.1 Pa·s at a temperature of 25° C. and at a shear rate of 1 s⁻¹.

The liquid fatty substances that can be used in the composition according to the invention are generally not oxyalkylenated and preferably do not contain any COOH carboxylic acid function.

Advantageously, the liquid fatty substances according to the invention may be chosen from hydrocarbons, fatty alcohols preferably comprising from 8 to 40 carbon atoms, fatty esters preferably comprising from 8 to 40 carbon atoms, fatty ethers preferably comprising from 8 to 40 carbon atoms, silicones and mixtures thereof.

By liquid hydrocarbon, it should be understood a hydrocarbon composed only of carbon and hydrogen atoms, liquid at a temperature of 25° C.) and at atmospheric pressure (760 mm Hg, namely 1.013×10⁵ Pa), of mineral or plant or synthetic origin.

More particularly, the liquid hydrocarbons are chosen from:
linear or branched, optionally cyclic, C₆-C₁₆ alkanes. For example, mention may be made of hexane, undecane, dodecane, tridecane and isoparaffins like isohexadecane, isododecane and isodecane, and mixtures thereof
linear or branched hydrocarbons, of mineral, animal or synthetic origin, with more than 16 carbon atoms, such as paraffin or Vaseline oils, Vaseline oil, polydecenes, hydrogenated polyisobutene such as that one sold under the Parleam® brand by the company NOF Corporation, squalane.

By liquid fatty alcohol, it should be understood a non-glycerolated and non-oxyalkylenated fatty alcohol, liquid at 30° C.) and at atmospheric pressure (760 mm Hg, namely 1.013×10⁵ Pa).

Preferably, the liquid fatty alcohols of the invention include from 8 to 30 carbon atoms, still better from 8 to 20 carbon atoms.

The liquid fatty alcohols of the invention may be saturated or unsaturated.

Preferably, the saturated liquid fatty alcohols are branched. They may possibly comprise in their structure at least one aromatic ring, or not. Preferably, they are acyclic.

More particularly, the liquid saturated fatty alcohols of the invention are chosen from octyldodecanol, isostearyl alcohol and 2-hexyldecanol.

The liquid unsaturated fatty alcohols have in their structure at least one double or triple bond, and preferably, one or more double bond(s). When several double bonds are present, they are preferably 2 or 3 in number and they may be conjugated, or not.

These unsaturated fatty alcohols may be linear or branched.

They may possibly comprise in their structure at least one aromatic ring, or not. Preferably, they are acyclic.

More particularly, the liquid unsaturated fatty alcohols of the invention are chosen from oleic (or oleyl) alcohol, linoleic (or linoleyl) alcohol, linolenic (or linolenyl) alcohol, and undecylenic alcohol.

By liquid fatty ester, it should be understood an ester derived from a fatty acid and/or a fatty alcohol, liquid at 30° C.) and at atmospheric pressure (760 mm Hg, namely 1.013×10⁵ Pa), and different from the fatty acid and (poly)glycerol monoester(s).

Preferably, the liquid fatty esters are the liquid esters of saturated or unsaturated, linear or branched, C₁-C₂₆ aliphatic mono- or polyacids and of saturated or unsaturated, linear or branched, C₁-C₂₆ aliphatic mono- or polyalcohols, the total number of carbon atoms of the liquid esters being greater than or equal to 10.

Preferably, for fatty esters of monoalcohols, at least one amongst the alcohol or acid from which the esters of the invention are derived is branched.

Among the monoesters of monoacids and monoalcohols, mention may be made of ethyl and isopropyl palmitates, alkyl myristates such as isopropyl or ethyl myristate, isocetyl stearate, ethyl laurate, 2-ethylhexyl isononanoate, isononyl isononanoate, ethyl octanoate, ethyl caprate, isodecyl neopentanoate, and isostearyl neopentanoate.

C₄-C₂₂ esters of di- or tri-carboxylic acids and C₁-C₂₂ alcohols and esters of mono-, di- or tri-carboxylic acids and C₄-C₂₆ di-, tri-, tetra- or pentahydroxylated non-sugar alcohols may also be used.

Finally, it is also possible to use natural or synthetic esters of di- or tri-acids with glycerol.

Among these, mention may be made of vegetable oils or oils of plant origin.

As oils of plant origin or synthetic triglycerides, which can be used in the composition of the invention as liquid fatty esters, mention may be made, for example, of triglyceride oils of plant or synthetic origin, such as liquid triglycerides of fatty acids including from 6 to 30 carbon atoms like the triglycerides of heptanoic or octanoic acids or, for example, sunflower, corn, soy, squash, grape seed, sesame, hazelnut, apricot, macadamia, arara, sunflower, castor, avocado, olive, rapeseed, copra, wheat germ, sweet almond, apricot, safflower, candlenut, camelina, tamanu, babassu and pracaxi oils, triglycerides of caprylic/capric acids like those sold by the company STEARINERIES DUBOIS or those sold under the names Miglyol® 810, 812 and 818 by the company DYNAMIT NOBEL, jojoba oil, shea butter oil.

The oil(s) that can be used in the composition according to the invention may also be chosen from silicones.

Preferably, the liquid silicone(s) are chosen from polydialkylsiloxanes, in particular polydimethylsiloxanes (PDMS), and organo-modified polysiloxanes including at least one functional group chosen from amino groups, aryl groups and alkoxy groups.

Organopolysiloxanes are defined in more detail in the work by Walter NOLL “Chemistry and Technology of Silicones” (1968), Academie Press.

The organomodified silicones that can be used in accordance with the invention are silicones as defined before and including in their structure one or more organofunctional group(s) fixed via a hydrocarbon group.

The organomodified silicones may be polydiaryl siloxanes, in particular polydiphenylsiloxanes, and polyalkyl-arylsiloxanes functionalised with the aforementioned organofunctional groups.

The liquid fatty substance(s) according to the invention are different from amitriptyline and the previously-described surfactants.

According to a particular embodiment of the invention, the composition further comprises at least one liquid fatty substance chosen from hydrocarbons, fatty esters comprising from 8 to 40 carbon atoms, silicones, and mixtures thereof.

Preferably, the total content of liquid fatty substance(s) is comprised between 0% and 20% by weight, more preferably between 0% and 10% by weight, with respect to the total weight of the composition.

More preferably, the composition according to the invention does not comprise any liquid fatty substance, different from amitriptyline and the previously-described surfactants.

The composition according to the invention may further contain additives commonly used in pharmaceuticals, like one or more perfume(s) and/or antibacterial(s).

As antibacterial, parabens, such as methyl-paraben or propyl-paraben, phenoxyethanol and isothiazolinones are preferably used, and more preferably isothiazolinones such as benzisothiazolinone, methylisothiazolinone and/or methylchloroisothiazolinone.

These additives may be present in the composition according to the invention in an amount ranging from 0% to 20% by weight with respect to the total weight of the composition.

A person skilled in the art will make sure that these possible additives and their amounts are selected so that they do not affect the properties of the compositions of the present invention.

The pH of the aqueous phase of the composition according to the invention is greater than or equal to 7.

Preferably, the pH of the aqueous phase is greater than or equal to 8, more preferably the pH ranges from 8 to 13, even more preferably from 8.5 to 13, still better from 9 to 12.5, and even better from 9 to 12.

According to a variant of the invention, the pH of the composition according to the invention is comprised between 7 and 8.

The pH may be adjusted to the desired value using commonly used alkalising agents. Among the alkalising agents, mention may be made, as examples, of ammonia, alkanolamines, mineral or organic hydroxides. The composition according to the invention may possibly comprise one or more alkalising agent(s) as mentioned hereinbefore.

To adjust the pH, the composition according to the invention may possibly comprise one or more buffer solution(s).

As an example, mention may be made of phosphate, citrate, borate, sorbate, acetate or tris-EDTA buffers. These buffers are marketed in particular by the companies Fischer or VWR.

According to a preferred embodiment of the invention, the composition comprises:
(i) an oily phase containing at least amitriptyline in its basic form, in a total content ranging from 1% to 10% by weight with respect to the total weight of the composition, preferably from 1% to 9% by weight, more preferably from 1% to 8% by weight, even more preferably from 1% to 7% by weight, with respect to the total weight of the composition; and
(ii) an aqueous phase, having a pH higher than or equal to 7.

According to a particular embodiment of the invention, the composition comprises:
(i) an oily phase consisting of amitriptyline in its basic form, in a total content ranging from 1% to 10% by weight with respect to the total weight of the composition, preferably from 1% to 9% by weight, more preferably from 1 to 8% by weight, even more preferably from 1% to 7% by weight, with respect to the total weight of the composition;
(ii) an aqueous phase, having a pH higher than or equal to 7.

According to a variant of the invention, the composition comprises:
(i) an oily phase containing at least amitriptyline in its basic form, in a total content ranging from 1% to 10% by weight with respect to the total weight of the composition, preferably from 1% to 9% by weight, more preferably from 1% to 8% by weight, even more preferably from 1% to 7% by weight, with respect to the total weight of the composition; and
(ii) an aqueous phase, having a pH comprised between 7 and 8.

The invention is a composition as described before for the topical use thereof. The composition according to the invention is a composition suited for topical administration.

Another object of the invention is also a composition according to the invention as described previously for use thereof as a medicine product.

Another object of the invention is also a method for treating neuropathic pain comprising the topical application of a pharmaceutical composition according to the invention as described previously; preferably for treating peripheral neuropathic pain; more preferably for treating chemotherapy-induced peripheral neuropathic pain, post-herpetic neuropathic pain, or diabetic neuropathic pain; even more preferably for treating chemotherapy-induced peripheral neuropathic pain.

Another object of the invention is a composition according to the invention as described before for the topical use thereof in the treatment of neuropathic pain; preferably for the topical use thereof in the treatment of peripheral neuropathic pain; more preferably for the topical use thereof in the treatment of chemotherapy-induced peripheral neuropathic pain, post-herpetic neuropathic pain, diabetic neuropathic pain; even more preferably for the topical use thereof in the treatment of chemotherapy-induced peripheral neuropathic pain.

Another object of the invention is also a method for treating erythromelalgia comprising the topical application of a pharmaceutical composition according to the invention as described previously.

Another object of the invention is a composition according to the invention as described before for the topical use thereof in the treatment of erythromelalgia.

The invention also relates to a method for remedying or preventing neuropathic pain likely to be induced by chemotherapy, comprising the topical application of a pharmaceutical composition according to the invention as described previously.

More particularly, the invention relates to a method for treating cancers including chemotherapy sessions, the pharmaceutical composition according to the invention as described previously being administered topically between the chemotherapy sessions to remedy or prevent neuropathic pain likely to be induced by chemotherapy.

The invention also relates to a composition according to the invention as described before for use thereof for remedying or preventing neuropathic pain likely to be induced by chemotherapy.

More particularly, the invention relates to a composition according to the invention as described before for use thereof in the treatment of cancers including chemotherapy sessions, the composition being administered topically between the chemotherapy sessions to remedy or prevent neuropathic pain likely to be induced by chemotherapy.

The following examples illustrate the composition according to the invention and the advantages of this composition. However, they in no way represent a limitation of the present invention but simply illustrate the invention.

### EXAMPLE

The composition A according to the following invention has been prepared from the ingredients indicated in the table hereinafter, the amounts of which are expressed in % by weight.

| COMPOSITION A (Invention) | Amount |
|---|---|
| Amitriptyline | 8 |
| Hydroxyethyl cellulose | 1 |
| Propylene glycol | 5 |
| Methylparaben | 0.1 |
| pH agent | Q.S. pH 7 |
| Water | Q.S. 100 |

In particular, it has been observed that an increase in the pH of a composition based on amitriptyline beyond 7 allows maintaining good effectiveness, and possibly improving effectiveness, in cutaneous application in the treatment of pain, while reducing the concentration of amitriptyline and thus reducing the risk of undesirable effects.

It has also been observed that the composition according to the invention provides good penetration of amitriptyline through the skin, even for a low content of amitriptyline.

1. A pharmaceutical composition in the form of an oil-in-water emulsion for topical application comprising:
(i) an oily phase containing at least amitriptyline in its basic form, in a total content ranging from 1% to 30% by weight with respect to the total weight of the composition; and
(ii) an aqueous phase, having a pH higher than or equal to 7.

2. The composition according to claim 1, characterised in that the total content of amitriptyline ranges from 1% to 10% by weight, with respect to the total weight of the composition.

3. The composition according to claim 2, characterised in that the total content of amitriptyline ranges from 1% to 9% by weight, with respect to the total weight of the composition.

4. The composition according to claim 3, characterised in that the total content of amitriptyline ranges from 1% to 8% by weight, more preferably from 1% to 7% by weight, with respect to the total weight of the composition.

5. The composition according to claim 1, characterised in that the volume-average size of the oily droplets ranges from 1 nm to 50 μm; preferably ranges from 10 nm to 20 μm.

6. The composition according to claim 5, characterised in that the volume-average size of the oily droplets ranges from 100 nm to 10 μm.

7. The composition according to claim 1, characterised in that the pH of the aqueous phase is higher than or equal to 8; preferably ranges from 8 to 13; more preferably from 8.5 to 13; even more preferably from 9 to 12.5; and even better from 9 to 12.

8. The composition according to claim 1, characterised in that it further comprises at least one surfactant; preferably chosen from non-ionic surfactants.

9. The composition according to claim 8, characterised in that it further comprises at least one non-ionic surfactant, optionally polyoxyalkylenated, chosen from sorbitan esters, glycerol esters, and mixtures thereof.

10. The composition according to claim 1, characterised in that it further comprises at least one C₂-C₈ polyol.

11. The composition according to claim 1, characterised in that it further comprises at least one thickening agent; preferably chosen from cellulose polymers.

12. The composition according to claim 1, characterised in that the water content is greater than or equal to 75% by weight, preferably comprised between 75% and 95% by weight; and more preferably comprised between 75% and 90% by weight, with respect to the total weight of the composition.

13. The composition according to claim 1, characterised in that amitriptyline in its basic form constitutes the oily phase.

14. The composition according to claim 1, characterised in that it comprises at least one liquid fatty substance; preferably chosen from hydrocarbons, fatty esters comprising from 8 to 40 carbon atoms, silicones, and mixtures thereof.

15. A method for treating neuropathic pain comprising the topical application of a pharmaceutical composition in the form of an oil-in-water emulsion for topical application comprising:
(iii) an oily phase containing at least amitriptyline in its basic form, in a total content ranging from 1% to 30% by weight with respect to the total weight of the composition; and
(iv) an aqueous phase, having a pH higher than or equal to 7.

16. The method according to claim 15, for treating chemotherapy-induced peripheral neuropathic pain, post-herpetic neuropathic pain or diabetic neuropathic pain; more preferably for treating chemotherapy-induced peripheral neuropathic pain.

17. A method for treating erythromelalgia comprising the topical application of a pharmaceutical composition in the form of an oil-in-water emulsion for topical application comprising:
(iii) an oily phase containing at least amitriptyline in its basic form, in a total content ranging from 1% to 30% by weight with respect to the total weight of the composition; and
(iv) an aqueous phase, having a pH higher than or equal to 7.
